(19) United States
(12) Patent Application Publication
(10) Pub. No.: US 2025/0188680 A1
(43) Pub. Date: Jun. 12, 2025

(54) COMPOSITIONS AND METHODS FOR PRODUCING FUNGAL TEXTILE MATERIAL

(71) Applicant: ARIZONA BOARD OF REGENTS ON BEHALF OF ARIZONA STATE UNIVERSITY, Scottsdale, AZ (US)

(72) Inventor: Benjamin GONZALEZ, Cottage Grove, OR (US)

(21) Appl. No.: 18/844,090

(22) PCT Filed: Mar. 9, 2023
(86) PCT No.: PCT/US2023/064011

§ 371 Date: Sep. 5, 2024

Related U.S. Application Data
(60) Provisional application No. 63/269,132, filed on Mar. 10, 2022.

(51) Int. Cl. D21H 13/30; C12N 1/14; D21H 17/02

(52) U.S. Cl. CPC D21H 13/30; C12N 1/14; D21H 17/02

(57) ABSTRACT

Described herein is a process in which filamentous fungi can be used to create textile or paper materials with tunable properties. The textiles can range from leather-like to paper-like depending on the species of fungi used and the growth conditions. The method of production utilizes the tendency of filamentous fungi to form films on still liquids, in order to generate many mycelial mats concurrently. These mats can then be layered to create textile materials, paper materials, and other material types having varying thickness, composition, and mechanical properties.

### CROSS-REFERENCE TO RELATED APPLICATIONS

This application claims priority to U.S. Provisional Patent Application No. 63/269,132, filed on Mar. 10, 2022, which is incorporated by reference in its entirety.

### TECHNICAL FIELD

Described herein is a process in which filamentous fungi can be used to create textile or paper materials with tunable properties. The textiles can range from leather-like to paper-like depending on the species of fungi used and the growth conditions. The method of production utilizes the tendency of filamentous fungi to form films on still liquids, in order to generate many mycelial mats concurrently. These mats can then be layered to create textile materials, paper materials, and other material types having varying thickness, composition, and mechanical properties.

### BACKGROUND

Currently, many faux leather materials are created using fungi by gradually growing layers or inoculating a solid substrate. Additionally, many current fungal biomaterials focus on the use of mushroom-forming species such as Pleurotus ostreatus. These approaches tend to be relatively slow, expensive, complex, and not readily scalable.

Thus, what is needed are new methods for generating various types of textiles or paper-like materials from fungal species that increases speed, decreases cost and complexity, and allows for easy scalability, without sacrificing any of the strengths of the fungal materials.

### SUMMARY

One embodiment described herein is a method for generating a textile or paper material from filamentous fungi, the method comprising: (a) growing a culture of filamentous fungi; (b) forming a plurality of mycelial mats; (c) harvesting the plurality of mycelial mats; and (d) combining the plurality of mycelial mats into a stack of mycelial mats. In one aspect, growing the culture of filamentous fungi comprises first growing on solid media to isolate conidia, followed by growing the isolated conidia in liquid media to generate a liquid culture. In another aspect, forming the plurality of mycelial mats comprises inoculating liquid media in a plurality of growth chambers with the liquid culture. In another aspect, the liquid culture is added to the liquid media in the plurality of growth chambers at a 1:10 volume ratio. In another aspect, the liquid media comprises a broth comprising yeast extract and malt extract. In another aspect, the plurality of mycelial mats is formed on the liquid media in the growth chambers at a temperature ranging from about 25° C. to about 50° C. over about 5 days to about 10 days. In another aspect, the plurality of mycelial mats is formed on the liquid media in the growth chambers at a temperature ranging from about 40° C. to about 45° C. over about 5 days to about 8 days. In another aspect, each growth chamber comprises a stainless-steel chamber comprising a depth of about 2 inches. In another aspect, the growth chamber comprises about 1 inch of liquid media throughout the chamber. In another aspect, each of the mycelial mats comprises a dry thickness of up to about 1 mm. In another aspect, the filamentous fungi comprise Neurospora crassa or Rhizopus oryzae. In another aspect, the stack of mycelial mats comprises from about 2 to about 16 individual mycelial mats. In another aspect, the stack of mycelial mats comprises from about 8 to about 16 individual mycelial mats. In another aspect, the method further comprises softening the stack of mycelial mats by treating with a plasticizer. In another aspect, treating with a plasticizer comprises submerging the stack of mycelial mats in a bath of plasticizer, wherein the plasticizer treats the outer surface of the stack and penetrates the interior of the stack to treat each of the plurality of mycelial mats. In another aspect, the bath of plasticizer comprises about 40% glycerol in water. In another aspect, a higher concentration of the plasticizer generates a softer and more malleable textile or paper material, and wherein a lower concentration of the plasticizer generates a harder and less malleable textile or paper material. In another aspect, the method further comprises crosslinking the stack of mycelial mats using a crosslinking agent selected from the group consisting of citric acid, calcium carbonate, paraformaldehyde, vegetable tannins, a mixture of citric acid and calcium carbonate, and combinations thereof. In another aspect, the crosslinking agent comprises a mixture of citric acid and calcium carbonate comprising about 10% citric acid and about 5 g/L calcium carbonate. In another aspect, the method further comprises processing the surface of the stack of mycelial mats to generate a textile or paper material with desired surface properties, wherein the desired surface properties include increased hydrophobicity or coloration. In another aspect, the surface of the stack of mycelial mats is treated with 100% vegetable oil to increase hydrophobicity. In another aspect, the surface of the stack of mycelial mats is treated with a color dye.

Another embodiment described herein is a textile or paper material from filamentous fungi made by any of the methods described herein.

Another embodiment described herein is a textile or paper material composition comprising a stack of filamentous fungi mycelial mats, made by a method comprising: (a) growing a culture of filamentous fungi; (b) forming a plurality of mycelial mats; (c) harvesting the plurality of mycelial mats; and (d) combining the plurality of mycelial mats into a stack of mycelial mats. In one aspect, the filamentous fungi comprise Neurospora crassa or Rhizopus oryzae. In another aspect, the textile material composition is leather-like when the filamentous fungi comprise Neurospora crassa. In another aspect, the textile material composition is cotton-like when the filamentous fungi comprise Rhizopus oryzae. In another aspect, the composition further comprises a plasticizer selected from the group consisting of mineral oil, vegetable oil, glycerol, and combinations thereof. In another aspect, the composition further comprises a crosslinking agent selected from the group consisting of citric acid, calcium carbonate, paraformaldehyde, vegetable tannins, a mixture of citric acid and calcium carbonate, and combinations thereof. In another aspect, the stack of filamentous fungi mycelial mats comprises from about 2 to about 16 individual mycelial mats. In another aspect, the stack of filamentous fungi mycelial mats comprises a thickness ranging from about 2 mm to about 16 mm.

Another embodiment described herein is a fungal textile or paper material comprising a stack of mycelial mats formed from the species Neurospora crassa, wherein the stack of mycelial mats comprises a crosslinking agent and a plasticizer.

Another embodiment described herein is a fungal textile or paper material comprising a stack of mycelial mats formed from the species Rhizopus oryzae, wherein the stack of mycelial mats comprises a crosslinking agent and a plasticizer.

In one aspect, the crosslinking agent comprises a mixture of citric acid and calcium carbonate.

In another aspect, the plasticizer comprises glycerol.

In another aspect, the stack of mycelial mats comprises a thickness ranging from about 2 mm to about 16 mm.

Another embodiment described herein is a growth chamber for cultivating fungi for producing mycelial mats as described herein, wherein the growth chamber comprises a stainless-steel chamber comprising a depth of about 2 inches, and wherein the growth chamber comprises about 1 inch of liquid media throughout the chamber.

Another embodiment described herein is a method for generating a fungal paper material from filamentous fungi, the method comprising: (a) growing a fungal biomass of filamentous fungi; (b) macerating the fungal biomass of filamentous fungi into a pulp; (c) straining the pulp to remove liquid; (d) incubating the pulp with NaOH; (e) neutralizing the pulp with acetic acid; (f) straining the pulp to remove liquid; (g) washing the pulp with water; (h) straining the pulp to remove liquid; (i) mixing the pulp with starch, CaCO₃, and glycerol to create a mixture; (j) pressing the mixture into one or more sheets of fungal paper material; (k) drying the one or more sheets of fungal paper material; (1) optionally, bleaching the one or more sheets of fungal paper material; and (m) optionally, cutting the one or more sheets of fungal paper material. In one aspect, the fungal biomass of filamentous fungi comprises Neurospora crassa or Rhizopus oryzae. In another aspect, incubating the pulp with NaOH comprises incubating at room temperature for about 30 min. In another aspect, washing the pulp with water comprises a ratio of water:pulp of about 1:2. In another aspect, mixing the pulp with starch, CaCO₃, and glycerol comprises a mixture ratio of about 75% pulp, about 10% starch, about 10% CaCO₃, and about 5% glycerol. In another aspect, drying the one or more sheets of fungal paper material comprises drying at about 85° C. for about 2 hours.

Another embodiment described herein is a fungal paper material from filamentous fungi made by any of the methods described herein.

### DESCRIPTION OF THE DRAWINGS

FIG. 1 shows a general process flow for creating a textile or paper material from filamentous fungi. The process comprises culturing the filamentous fungi on solid medium (100); isolating spores of the filamentous fungi (102); inoculating liquid cultures with the isolated spores (104); plating and growing liquid cultures in trays (106); harvesting a plurality of mycelial mats (108); combining the plurality of mycelial mats to produce a multi-layered (i.e., stacked) mycelial mat (110); and performing any additional processing techniques to form a stacked mycelial mat with desired surface properties including increased hydrophobicity or coloration.

FIG. 2 shows a chamber precursor model where a stacking petri dish method (200) was used to create fungal leather samples. Individual petri dish plates (202) were combined together to create a stacked fungal leather sample (204).

FIG. 3 shows a growth chamber model (“Chamber V1”) (300).

FIG. 4 shows a growth chamber model (“Chamber V2”) (400).

FIG. 5 shows a growth chamber model (“Chamber V3”) (500).

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.” All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “textile” or “textile material” refer to fungal material (e.g., from filamentous fungi) that resembles cloth-like, woven fabric, and fiber-based textile materials, including fibers, yarns, filaments, threads, different fabric types, and the like. These fungal textiles or fungal textile materials may resemble the cloth-like, woven fabric, and fiber-based textile materials in their structural, visual, compositional, mechanical, and/or textural properties, as well as in any other properties or characteristics. In some non-limiting embodiments of the present invention, fungal textile material compositions may be leather-like when the filamentous fungi comprise Neurospora crassa. In other non-limiting embodiments of the present invention, fungal textile material compositions may be cotton-like when the filamentous fungi comprise Rhizopus oryzae.

As used herein, the terms “paper” or “paper material” refer to fungal material (e.g., from filamentous fungi) that resembles paper derived from softwood or hardwood trees (i.e., paper derived from wood pulp). This fungal paper or fungal paper material may resemble the paper derived from softwood or hardwood trees in its structural, visual, compositional, mechanical, and/or textural properties, as well as in any other properties or characteristics.

As used herein, the term “mycelial mat” refers to a layer of fungal biomass derived from growing and culturing one or more types of filamentous fungi in a specific apparatus designed to produce and maintain such a layer of fungal biomass by cultivating the filamentous fungi. In some non-limiting embodiments of the present invention, mycelial mats may be produced in a growth chamber comprising a stainless-steel chamber comprising a depth of about 2 inches, where the growth chamber comprises about 1 inch of liquid media throughout the chamber.

Described herein is essentially a process for creating large amounts of biofilms using filamentous fungi that are typically thought of as molds. This process allows for rapid production of these biofilms in a manner that is easily scalable and adjustable to create a variety of cheap biomaterials. These biomaterials can range from hard plastic-like materials to leather- or cotton-like textiles. This process also utilizes the natural growth characteristics and biochemistry of fungi to allow post processing that further tunes the properties of the material. Using the right species of fungi and the right processing techniques can allow for rapid and cheap materials to be produced like faux leather, soft cotton-like fabrics, spools of threads, hard plastic-like sheets, or paper-like mats. The described processes can easily be made to be non-toxic and environmentally friendly because they utilize the natural growth and biochemistry of filamentous fungi.

Some of the advantageous aspects of the described technology include the speed, tunability, and specific starting materials for the production of fungal textiles. Fungal textiles have been created before; however, these “molds” grow quickly, naturally form films on still liquid surfaces, and can have varying properties depending on species and conditions. Filamentous fungi naturally crosslink with each other to a varying degree, depending on the particular fungal species. This can be exploited to create materials that are more fibrous and cotton-like, or tough and leather-like. Additionally, growing several films concurrently in large trays allows for very rapid production of fungal textiles using very cheap starting materials, such as potato broth or lysogeny broth (LB). LB is a standard broth used in microbiology and potato broth is a standard broth used in mycology. Potato broth can also be referred to as potato-dextrose broth if it is supplemented with dextrose.

The described processes explore the use of crosslinking agents for the carbohydrate chitin, such as citric acid and calcium carbonate, to provide a non-toxic alternative to traditional leather crosslinking agents that would otherwise not be available. The described processes also allow for the tuning of the malleability of the fungal material by adjusting the concentration of a plasticizer, which opens the possibility for creating hard plastic-like surfaces. Plasticizers are used in plastic formation to reduce intermolecular forces and make plastics more flexible and less brittle. Plasticizers can be used with the described fungal leather biomaterials, which are largely composed of biopolymers, namely chitin, glucans, and fungal proteins and lipids.

Currently, many faux leather materials are created using fungi by gradually growing layers or inoculating a solid substrate. These approaches tend to be relatively slow, expensive, complex, and not readily scalable. The described processes improve on these other methods by growing mycelial films (i.e., mats) using nothing but the liquid media itself and doing so many times simultaneously. This increases speed and decreases cost and complexity, while making scaling the process easier. Many current fungal biomaterials also focus on the use of mushroom-forming species such as Pleurotus ostreatus; however, fungal species such as Neurospora crassa and Rhizopus oryzae grow very fast and are easier to culture without sacrificing any of the strength of fungal materials.

The described methods of creating fungal textile materials from molds have several advantages over other methods used to create fungal leather materials: (1) The use of mold species takes advantage of high growth rates and high anastomoses rates that produce stronger leather in a shorter amount of time; (2) the methods described herein use still liquid media as the growth medium which is very low cost and can be made in-house from common materials such as agricultural waste or fermentation waste; (3) the material properties and the fungal growth rates can be tailored by using different fungal species and environmental factors, allowing for the creation of other types of materials besides faux leather, including hard plastic-like materials and fibrous cotton-like textiles; and (4) the process is easily scalable and can be varied depending on the desired material.

One embodiment described herein is a method for generating a textile or paper material from filamentous fungi, the method comprising: (a) growing a culture of filamentous fungi; (b) forming a plurality of mycelial mats; (c) harvesting the plurality of mycelial mats; and (d) combining the plurality of mycelial mats into a stack of mycelial mats. In one aspect, growing the culture of filamentous fungi comprises first growing on solid media to isolate conidia, followed by growing the isolated conidia in liquid media to generate a liquid culture. In another aspect, forming the plurality of mycelial mats comprises inoculating liquid media in a plurality of growth chambers with the liquid culture. In another aspect, the liquid culture is added to the liquid media in the plurality of growth chambers at a 1:10 volume ratio. In another aspect, the liquid media comprises a broth comprising yeast extract and malt extract. In another aspect, the plurality of mycelial mats is formed on the liquid media in the growth chambers at a temperature ranging from about 10° C. to about 70° C. over about 3 days to about 20 days. In another aspect, the plurality of mycelial mats is formed on the liquid media in the growth chambers at a temperature ranging from about 10° C. to about 70° C. over about 5 days to about 10 days. In another aspect, the plurality of mycelial mats is formed on the liquid media in the growth chambers at a temperature ranging from about 25° C. to about 50° C. over about 3 days to about 20 days. In another aspect, the plurality of mycelial mats is formed on the liquid media in the growth chambers at a temperature ranging from about 25° C. to about 50° C. over about 5 days to about 10 days. In another aspect, the plurality of mycelial mats is formed on the liquid media in the growth chambers at a temperature ranging from about 40° C. to about 45° C. over about 5 days to about 8 days. In another aspect, each growth chamber comprises a stainless-steel chamber comprising a depth of about 2 inches. In another aspect, the growth chamber comprises about 1 inch of liquid media throughout the chamber. In another aspect, each of the mycelial mats comprises a dry thickness ranging from about 0.1 mm to about 1 mm. In another aspect, each of the mycelial mats comprises a dry thickness of up to about 1 mm. In another aspect, each of the mycelial mats comprises a dry thickness greater than about 1 mm. In another aspect, the filamentous fungi comprise Neurospora crassa or Rhizopus oryzae. In another aspect, the stack of mycelial mats comprises from about 2 to about 30 individual mycelial mats. In another aspect, the stack of mycelial mats comprises from about 2 to about 16 individual mycelial mats. In another aspect, the stack of mycelial mats comprises from about 8 to about 16 individual mycelial mats. In another aspect, the method further comprises softening the stack of mycelial mats by treating with a plasticizer. In another aspect, treating with a plasticizer comprises submerging the stack of mycelial mats in a bath of plasticizer, wherein the plasticizer treats the outer surface of the stack and penetrates the interior of the stack to treat each of the plurality of mycelial mats. In another aspect, the bath of plasticizer comprises about 30% to about 50% glycerol in water. In another aspect, the bath of plasticizer comprises about 40% glycerol in water. In another aspect, a higher concentration of the plasticizer generates a softer and more malleable textile or paper material, and wherein a lower concentration of the plasticizer generates a harder and less malleable textile or paper material. In another aspect, the method further comprises crosslinking the stack of mycelial mats using a crosslinking agent selected from the group consisting of citric acid, calcium carbonate, paraformaldehyde, vegetable tannins, a mixture of citric acid and calcium carbonate, and combinations thereof. In another aspect, the crosslinking agent comprises a mixture of citric acid and calcium carbonate comprising about 5% to about 15% citric acid and about 2.5 g/L to about 7.5 g/L calcium carbonate. In another aspect, the crosslinking agent comprises a mixture of citric acid and calcium carbonate comprising about 10% citric acid and about 5 g/L calcium carbonate. In another aspect, the method further comprises processing the surface of the stack of mycelial mats to generate a textile or paper material with desired surface properties, wherein the desired surface properties include increased hydrophobicity or coloration. In another aspect, the surface of the stack of mycelial mats is treated with 100% vegetable oil to increase hydrophobicity. In another aspect, the surface of the stack of mycelial mats is treated with a color dye.

Another embodiment described herein is a textile or paper material from filamentous fungi made by any of the methods described herein.

Another embodiment described herein is a textile or paper material composition comprising a stack of filamentous fungi mycelial mats, made by a method comprising: (a) growing a culture of filamentous fungi; (b) forming a plurality of mycelial mats; (c) harvesting the plurality of mycelial mats; and (d) combining the plurality of mycelial mats into a stack of mycelial mats. In one aspect, the filamentous fungi comprise Neurospora crassa or Rhizopus oryzae. In another aspect, the textile material composition is leather-like when the filamentous fungi comprise Neurospora crassa. In another aspect, the textile material composition is cotton-like when the filamentous fungi comprise Rhizopus oryzae. In another aspect, the composition further comprises a plasticizer selected from the group consisting of mineral oil, vegetable oil, glycerol, and combinations thereof. In another aspect, the composition further comprises a crosslinking agent selected from the group consisting of citric acid, calcium carbonate, paraformaldehyde, vegetable tannins, a mixture of citric acid and calcium carbonate, and combinations thereof. In another aspect, the stack of filamentous fungi mycelial mats comprises from about 2 to about 30 individual mycelial mats. In another aspect, the stack of filamentous fungi mycelial mats comprises from about 2 to about 16 individual mycelial mats. In another aspect, each of the mycelial mats comprises a dry thickness ranging from about 0.1 mm to about 1 mm. In another aspect, each of the mycelial mats comprises a dry thickness of up to about 1 mm. In another aspect, each of the mycelial mats comprises a dry thickness greater than about 1 mm. In another aspect, the stack of filamentous fungi mycelial mats comprises a thickness ranging from about 2 mm to about 30 mm. In another aspect, the stack of filamentous fungi mycelial mats comprises a thickness ranging from about 2 mm to about 16 mm.

Another embodiment described herein is a fungal textile or paper material comprising a stack of mycelial mats formed from the species Neurospora crassa, wherein the stack of mycelial mats comprises a crosslinking agent and a plasticizer.

Another embodiment described herein is a fungal textile or paper material comprising a stack of mycelial mats formed from the species Rhizopus oryzae, wherein the stack of mycelial mats comprises a crosslinking agent and a plasticizer.

In one aspect, the crosslinking agent comprises a mixture of citric acid and calcium carbonate.

In another aspect, the plasticizer comprises glycerol.

In another aspect, the stack of mycelial mats comprises a thickness ranging from about 2 mm to about 30 mm. In another aspect, the stack of mycelial mats comprises a thickness ranging from about 2 mm to about 16 mm.

Another embodiment described herein is a growth chamber for cultivating fungi for producing mycelial mats as described herein, wherein the growth chamber comprises a stainless-steel chamber comprising a depth of about 2 inches, and wherein the growth chamber comprises about 1 inch of liquid media throughout the chamber.

Another embodiment described herein is a method for generating a fungal paper material from filamentous fungi, the method comprising: (a) growing a fungal biomass of filamentous fungi; (b) macerating the fungal biomass of filamentous fungi into a pulp; (c) straining the pulp to remove liquid; (d) incubating the pulp with NaOH; (e) neutralizing the pulp with acetic acid; (f) straining the pulp to remove liquid; (g) washing the pulp with water; (h) straining the pulp to remove liquid; (i) mixing the pulp with starch, CaCO₃, and glycerol to create a mixture; (j) pressing the mixture into one or more sheets of fungal paper material; (k) drying the one or more sheets of fungal paper material; (1) optionally, bleaching the one or more sheets of fungal paper material; and (m) optionally, cutting the one or more sheets of fungal paper material. In one aspect, the fungal biomass of filamentous fungi comprises Neurospora crassa or Rhizopus oryzae. In another aspect, incubating the pulp with NaOH comprises incubating at about 20° C. to about 30° C. for about 20 to about 40 minutes. In another aspect, incubating the pulp with NaOH comprises incubating at room temperature for about 20 to about 40 minutes. In another aspect, incubating the pulp with NaOH comprises incubating at room temperature for about 30 minutes. In another aspect, washing the pulp with water comprises a ratio of water:pulp of about 1:2. In another aspect, mixing the pulp with starch, CaCO₃, and glycerol comprises a mixture ratio of about 75% pulp, about 10% starch, about 10% CaCO₃, and about 5% glycerol. In another aspect, drying the one or more sheets of fungal paper material comprises drying at about 75° C. to about 95° C. for about 1.5 hours to about 3 hours. In another aspect, drying the one or more sheets of fungal paper material comprises drying at about 85° C. for about 2 hours.

Another embodiment described herein is a fungal paper material from filamentous fungi made by any of the methods described herein.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for generating a textile or paper material from filamentous fungi, the method comprising:
   (a) growing a culture of filamentous fungi;
   (b) forming a plurality of mycelial mats;
   (c) harvesting the plurality of mycelial mats; and
   (d) combining the plurality of mycelial mats into a stack of mycelial mats.
Clause 2. The method of clause 1, wherein growing the culture of filamentous fungi comprises first growing on solid media to isolate conidia, followed by growing the isolated conidia in liquid media to generate a liquid culture.
Clause 3. The method of clause 1 or 2, wherein forming the plurality of mycelial mats comprises inoculating liquid media in a plurality of growth chambers with the liquid culture.
Clause 4. The method of any one of clauses 1-3, wherein the liquid culture is added to the liquid media in the plurality of growth chambers at a 1:10 volume ratio.
Clause 5. The method of any one of clauses 1-4, wherein the liquid media comprises a broth comprising yeast extract and malt extract.
Clause 6. The method of any one of clauses 1-5, wherein the plurality of mycelial mats is formed on the liquid media in the growth chambers at a temperature ranging from about 25° C. to about 50° C. over about 5 days to about 10 days.
Clause 7. The method of any one of clauses 1-6, wherein the plurality of mycelial mats is formed on the liquid media in the growth chambers at a temperature ranging from about 40° C. to about 45° C. over about 5 days to about 8 days.
Clause 8. The method of any one of clauses 1-7, wherein each growth chamber comprises a stainless-steel chamber comprising a depth of about 2 inches.
Clause 9. The method of any one of clauses 1-8, wherein the growth chamber comprises about 1 inch of liquid media throughout the chamber.
Clause 10. The method of any one of clauses 1-9, wherein each of the mycelial mats comprises a dry thickness of up to about 1 mm.
Clause 11. The method of any one of clauses 1-10, wherein the filamentous fungi comprise Neurospora crassa or Rhizopus oryzae.
Clause 12. The method of any one of clauses 1-11, wherein the stack of mycelial mats comprises from about 2 to about 16 individual mycelial mats.
Clause 13. The method of any one of clauses 1-12, wherein the stack of mycelial mats comprises from about 8 to about 16 individual mycelial mats.
Clause 14. The method of any one of clauses 1-13, further comprising softening the stack of mycelial mats by treating with a plasticizer.
Clause 15. The method of any one of clauses 1-14, wherein treating with a plasticizer comprises submerging the stack of mycelial mats in a bath of plasticizer, wherein the plasticizer treats the outer surface of the stack and penetrates the interior of the stack to treat each of the plurality of mycelial mats.
Clause 16. The method of any one of clauses 1-15, wherein the bath of plasticizer comprises about 40% glycerol in water.
Clause 17. The method of any one of clauses 1-16, wherein a higher concentration of the plasticizer generates a softer and more malleable textile or paper material, and wherein a lower concentration of the plasticizer generates a harder and less malleable textile or paper material.
Clause 18. The method of any one of clauses 1-17, further comprising crosslinking the stack of mycelial mats using a crosslinking agent selected from the group consisting of citric acid, calcium carbonate, paraformaldehyde, vegetable tannins, a mixture of citric acid and calcium carbonate, and combinations thereof.
Clause 19. The method of any one of clauses 1-18, wherein the crosslinking agent comprises a mixture of citric acid and calcium carbonate comprising about 10% citric acid and about 5 g/L calcium carbonate.
Clause 20. The method of any one of clauses 1-19, further comprising processing the surface of the stack of mycelial mats to generate a textile or paper material with desired surface properties, wherein the desired surface properties include increased hydrophobicity or coloration.
Clause 21. The method of any one of clauses 1-20, wherein the surface of the stack of mycelial mats is treated with 100% vegetable oil to increase hydrophobicity.
Clause 22. The method of any one of clauses 1-21, wherein the surface of the stack of mycelial mats is treated with a color dye.
Clause 23. A textile or paper material from filamentous fungi made by the method of any one of clauses 1-22.
Clause 24. A textile or paper material composition comprising a stack of filamentous fungi mycelial mats, made by a method comprising:
   (a) growing a culture of filamentous fungi;
   (b) forming a plurality of mycelial mats;
   (c) harvesting the plurality of mycelial mats; and
   (d) combining the plurality of mycelial mats into a stack of mycelial mats.
Clause 25. The composition of clause 24, wherein the filamentous fungi comprise Neurospora crassa or Rhizopus oryzae.
Clause 26. The composition of clause 24 or 25, wherein the textile material composition is leather-like when the filamentous fungi comprise Neurospora crassa.
Clause 27. The composition of any one of clauses 24-26, wherein the textile material composition is cotton-like when the filamentous fungi comprise Rhizopus oryzae.
Clause 28. The composition of any one of clauses 24-27, further comprising a plasticizer selected from the group consisting of mineral oil, vegetable oil, glycerol, and combinations thereof.
Clause 29. The composition of any one of clauses 24-28, further comprising a crosslinking agent selected from the group consisting of citric acid, calcium carbonate, paraformaldehyde, vegetable tannins, a mixture of citric acid and calcium carbonate, and combinations thereof.
Clause 30. The composition of any one of clauses 24-29, wherein the stack of filamentous fungi mycelial mats comprises from about 2 to about 16 individual mycelial mats.
Clause 31. The composition of any one of clauses 24-30, wherein the stack of filamentous fungi mycelial mats comprises a thickness ranging from about 2 mm to about 16 mm.
Clause 32. A fungal textile or paper material comprising a stack of mycelial mats formed from the species Neurospora crassa, wherein the stack of mycelial mats comprises a crosslinking agent and a plasticizer.
Clause 33. A fungal textile or paper material comprising a stack of mycelial mats formed from the species Rhizopus oryzae, wherein the stack of mycelial mats comprises a crosslinking agent and a plasticizer.
Clause 34. The fungal textile or paper material of clause 32 or 33, wherein the crosslinking agent comprises a mixture of citric acid and calcium carbonate.
Clause 35. The fungal textile or paper material of any one of clauses 32-34, wherein the plasticizer comprises glycerol.
Clause 36. The fungal textile or paper material of any one of clauses 32-35, wherein the stack of mycelial mats comprises a thickness ranging from about 2 mm to about 16 mm.
Clause 37. A growth chamber for cultivating fungi for producing mycelial mats, wherein the growth chamber comprises a stainless-steel chamber comprising a depth of about 2 inches, and wherein the growth chamber comprises about 1 inch of liquid media throughout the chamber.
Clause 38. A method for generating a fungal paper material from filamentous fungi, the method comprising:
   (a) growing a fungal biomass of filamentous fungi;
   (b) macerating the fungal biomass of filamentous fungi into a pulp;
   (c) straining the pulp to remove liquid;
   (d) incubating the pulp with NaOH;
   (e) neutralizing the pulp with acetic acid;
   (f) straining the pulp to remove liquid;
   (g) washing the pulp with water;
   (h) straining the pulp to remove liquid;
   (i) mixing the pulp with starch, CaCO₃, and glycerol to create a mixture;
   (j) pressing the mixture into one or more sheets of fungal paper material;
   (k) drying the one or more sheets of fungal paper material;
   (l) optionally, bleaching the one or more sheets of fungal paper material; and
   (m) optionally, cutting the one or more sheets of fungal paper material.
Clause 39. The method of clause 38, wherein the fungal biomass of filamentous fungi comprises Neurospora crassa or Rhizopus oryzae.
Clause 40. The method of clause 38 or 39, wherein incubating the pulp with NaOH comprises incubating at room temperature for about 30 min.
Clause 41. The method of any one of clauses 38-40, wherein washing the pulp with water comprises a ratio of water:pulp of about 1:2.
Clause 42. The method of any one of clauses 38-41, wherein mixing the pulp with starch, CaCO₃, and glycerol comprises a mixture ratio of about 75% pulp, about 10% starch, about 10% CaCO₃, and about 5% glycerol.
Clause 43. The method of any one of clauses 38-42, wherein drying the one or more sheets of fungal paper material comprises drying at about 85° C. for about 2 hours.
Clause 44. A fungal paper material from filamentous fungi made by the method of any one of clauses 38-43.

### EXAMPLES

### Example 1

Chitin Extract from Agaricus bisporus

Approximately 0.5 kg (1 lb.) of A. bisporus mushrooms were used for chitin extraction.

The mushrooms were blended in both cold and hot water, and the resulting pulps were strained in cheese cloths to isolate the chitin-containing portion. Cold water was found to yield more chitin extract. The strained material was soaked in 1% Alconox® detergent solution (weight/volume) (e.g., sodium dodecyl benzenesulfonate, tetrasodium diphosphate, sodium carbonate), strained in cheese cloth, and then soaked in a 6% sodium hypochlorite (bleach) solution before a final straining. The resulting material was then placed in a 2.7% NaOH solution to partially de-acetylate the chitin and help solubilize the chitin while removing other proteins and lipids. The pulpy material was strained and washed with deionized water between each step. The chitin-containing material was again strained in cheese cloths and produced an off-white paste that was used for experiments on crosslinking and plasticizing. The resulting chitin pulp was then flattened and dried at 85° C., creating sheets of a desired thickness. Exemplary extraction conditions that were tested are shown below in Table 1.

TABLE 1
Chemical Concentrations for Chitin Extraction

| | Concentrations tested | |
|---|---|---|
| Extraction reagent | (weight/volume) | Optimal concentration |
| NaClO (Bleach) | 1%, 3%, 6%, 10% | 6%. This removed many contaminants |
| | | such as soils, proteins, and lipids from |
| | | the mushroom but left a large amount of |
| | | the chitin undissolved and also |
| | | increased yield. |
| Alconox ® (sodium | 0%, 1%, 10% | 1%. Washing with Alconox ® or any |
| dodecyl | | other type of soap removed |
| benzenesulfonate, | | contaminants, but higher concentrations |
| tetrasodium | | were too difficult to remove from the |
| diphosphate, sodium | | chitin material. |
| carbonate) | | |
| NaOH | 2.7%, 4%, 5% | 2.7%. NaOH removed many |
| | | contaminants and helped the chitin |
| | | suspend in solution by partial de- |
| | | acetylation of the polymer and |
| | | disruption of the hydrogen bonds |
| | | holding the chitin stacks together. |
| | | Higher concentrations completely |
| | | dissolved the chitin as free chitosan, |
| | | which was too difficult to recover |
| | | through precipitation. The low 2.7% |
| | | concentration was not enough to |
| | | completely convert chitin as is done in |
| | | shrimp chitin, but helped remove many |
| | | contaminants, put chitin in suspension |
| | | in the wash solution, and resulted in a |
| | | purer chitin material that could be |
| | | captured with straining. This low |
| | | concentration also may have improved |
| | | citric acid crosslinking by partially |
| | | deacetylating the chitin polymers, |
| | | opening it up for crosslinking. |

### Crosslinking and Plasticizing Chitin Extract

In order to make a solid textile material out of extracted chitin pulp, different crosslinking agents and plasticizers were used, as well as other additives to change the color and surface textures. The crosslinking agents that were tested included paraformaldehyde (PFA), CaCO₃, and citric acid. The plasticizers tested were mineral oil, vegetable oil, and glycerin. Chitin pulps were flattened into sheets and weighed down with metal trays while drying at 85° C. This drying temperature was chosen because it was not high enough to destroy the chitin, but still rapidly dried the chitin sheets. The resulting dried sheets were then soaked in varying concentrations of PFA, citric acid, and CaCO₃ to undergo crosslinking, as shown below in Table 2. All crosslinking occurred at room temperature over 24 hours which is standard practice.

TABLE 2
Chemical Concentrations for Chitin Crosslinking

| Crosslinking | Concentrations tested | |
|---|---|---|
| reagent | (% = weight/volume) | Optimal concentration |
| PFA | 2%, 4%, 10% | 10%. The toughness of the final material using this |
| | | high concentration was superior to all other |
| | | crosslinkers tested, except for the citric acid/CaCO₃ |
| | | combination, which was found to be equally |
| | | effective. |
| Citric acid | 2%, 4%, 10% | 10%. The final material was tougher to tear with |
| | | this high concentration. |
| CaCO₃ | 2 g/L, 4 g/L, 5 g/L | 5 g/L. The high concentration was optimal, but all |
| | | tests with CaCO₃ alone were generally less |
| | | effective than the other crosslinkers tested. |
| Citric | 10% citric acid; | This combination of citric acid with CaCO₃ was |
| acid/CaCO₃ | 5 g/L CaCO₃ | found to effectively crosslink the leather in a |
| combination | | manner comparable to the PFA, based on attempts |
| | | to tear the material. The citric acid likely created |
| | | an acidic environment that allowed for additional |
| | | CaCO₃ crosslinking to occur, which has been seen |
| | | in acid crosslinking of hydrogels. This crosslinking |
| | | method also has non-toxic properties. |

After crosslinking, various reagents were tested to increase flexibility, both by directly adding them to the pulp, adding as a surface coating, and adding within water baths. Mineral oil, mineral oil/Clear Rite 3™, glycerol, glycerol/water, and vegetable oil were the plasticizers tested to increase flexibility, as shown below in Table 3.

TABLE 3
Chemical Concentrations for Chitin Plasticization

| Plasticizing | Concentrations tested | |
|---|---|---|
| reagent | (% = weight/volume) | Optimal concentration |
| Mineral oil | 100% mixed in as a 1:4 | Not used because it did not penetrate as a coating |
| | ratio and as a surface | and separated when mixed. |
| | coating | |
| Vegetable oil | 100% mixed in as a 1:4 | Not used because it did not penetrate as a coating |
| | ratio and as a surface | and separated when mixed. |
| | coating | |
| Mineral oil/ | 50% mineral oil with | Not used because it was unable to penetrate the |
| Clear Rite 3 ™ | 50% Clear Rite 3 ™ | chitin and deliver the mineral oil. |
| | added as a surface | |
| | coating | |
| Glycerol | 1:1, 1:4, 1:10 ratios | 1:10 ratio. Was found to soften the material and |
| | mixed in and 100% as a | create a very flexible material that was very weak. |
| | surface coating | The surface coating approach did not penetrate |
| | | the chitin and was not used. |
| Glycerol/water | Added as 5%, 10%, | 40%. This method was found to be the best |
| | 20%, 40%, 50% | method for plasticizing and creating a strong |
| | concentrations of | flexible material. The material was soaked in a |
| | glycerol in water; the | bath at 60° C. for 30 min and the material was air- |
| | material was soaked | dried at room temperature. Heating near 85° C. |
| | while heating at approx. | causes the glycerol to evaporate, while room |
| | 60° C. | temperature to about 50° C. was found to be a |
| | | sufficient range to dry. The 40% glycerol/water |
| | | solution was reusable for other batches. |

Clear Rite 3™ (Fisher Scientific) is an organic solvent like Xylene and was tested as a mechanism for incorporating mineral oil into the material. Ultimately, a glycerol/water combination was found to work best where glycerol was added to the material in varying concentrations as a hot water/glycerol bath to increase the flexibility of the material. The final material may optionally receive additional surface coating treatments to seal and waterproof. The final surface treatments for sealing and waterproofing that were tested included CaCO₃, mineral oil, and vegetable oil, as shown below in Table 4.

TABLE 4
Chemical Concentrations for Optional Surface Coating

| Surface coating | Concentrations tested | |
|---|---|---|
| reagent | (% = weight/volume) | Results |
| Vegetable oil | Rubbed on the exterior at | This was the best method and left an oily |
| | 100%. | hydrophobic surface on the material when |
| | | removed, similar to leather oils. |
| Mineral oil | Rubbed on the exterior at | This coating did not remain on the material |
| | 100% | very well and was abandoned. The mineral |
| | | oil did not seem to mix well with chitin. |
| CaCO₃ | Painted onto the surface | This method created a white coating but |
| | as a saturated slurry at | penetrated the material too much and caused |
| | about 1 g/5 mL | it to become brittle and flakey. |

### Example 2

### Variations in Material Thickness for Increased Strength

Once the chemical conditions were optimized for A. bisporus mushroom pulp, leather material of different thickness was investigated. Generally, it was found that thicker pulp created stronger material. However, once the material was near about 15 mm thick, the material would tear while folding similar to an eraser being folded. Stacking of the material did not improve the strength and the large thick mats that were created would begin to break apart under high flexing and bending. This likely occurred because the natural interconnected tissue structure of the fungal mycelia and mushroom were lost during processing. Chitinous pulp may be better for a different type of material such as molded packaging material since it easily took on shapes, but the chemistry involved was used for the leather- and paper-like materials.

Large batches of mold cultures were then used as the starting material instead of A. bisporus mushrooms. During culturing, it was observed that both Neurospora crassa and Rhizopus oryzae created bio-mats at the liquid air interface when the water was still. Normally these species are cultured in shaking flasks to continuously aerate the media and fungus, but the still flasks grew these mats that thickened over time. The bio-mats were processed similar to the chitin pulp but were not blended and were instead kept in their natural tissue structure. Neurospora crassa was found to create a leather-like material and Rhizopus oryzae created a fibrous, paper-like, or cotton-like material. These observed differences may be due to the interconnection differences of the fungal mycelia, which is termed anastomosis, and this influenced the final materials. Thicker layers of the fungal bio-mats were then grown using different methods, media, growth conditions and chambers, and inoculation methods.

The bio-mats eventually leveled off in their thickness when allowed to grow over time, and it was found that after a week of growth, no significant change in thickness occurred. However, the final material needed to be thicker to be useful as a textile material. To encourage thicker growth, the fungal bio-mats were periodically submerged in new media, the bio-mats were flipped, the bio-mats were stacked with other fungal bio-mats grown separately, or the bio-mats were submerged in original media. Submerging the bio-mat in new media formed a new bio-mat at the air-liquid interface, and the submerged bio-mat began to deteriorate underneath. Flipping the bio-mat resulted in a new air-liquid interface and a brand-new bio-mat formed while the old bio-mat deteriorated. Submerging the bio-mat in the original media resulted in a new bio-mat at the new air-liquid interface, while the old bio-mat deteriorated. Finally, stacking together bio-mats that were grown separately resulted in a strong bonding of the bio-mats as well as a thicker leather material. This parallel growth of several bio-mats was then further explored because it generated the desired thickness of textile without the deterioration of any of the precursor fungal bio-mat material. The number of bio-mats needed for stacking was dependent on the strength desired.

After comparing stacks of 1, 2, 4, 8, and 16 bio-mats, it was found that 8-16 bio-mats stacked was a good range for a leather-like strength. Eight layers was sufficient, but more layers increased the strength of the material.

### Example 3

### Growth Media and Conditions for Fungal Bio-Mat Production Growth Media

Typical media used to grow the individual fungal species were originally used; however, some of these media formulations were expensive to create and tedious to make. Therefore, different media was tested to see if there was any material variance between different media. It is also preferable to only use one media to grow the materials for simplicity in production when creating different types of materials in large-scale. The media that were tested for Neurospora crassa and Rhizopus oryzae species are shown below in Table 5. Based on the observed results and known nutrient requirements of the two mold species, the potato dextrose broth and the malt extract broth were both supplemented with the yeast extract to create a cheap medium that still produced sufficient levels of growth. The yeast/malt extract broth was the preferred media overall.

TABLE 5
Fungal Growth Media for Bio-mat Production

| Media | Results |
|---|---|
| Vogel's complete | Yielded the most growth for both species, |
| medium | but it is the most expensive media. |
| Potato dextrose | Yielded the least growth for both species, |
| broth | but it is the cheapest media. |
| Yeast/Malt | Yielded a similar amount of growth as Vogel's |
| extract broth | complete medium and is similarly inexpensive |
| | when compared to potato dextrose broth. |
| Malt extract broth | Yielded growth similar to potato dextrose broth. |

### Growth Temperature and Humidity

The temperatures used during the growth of the bio-mats was optimized at a range of about 40-45° C. for rapid growth. Growth at about 40-45° C. was completed in about 5-8 days. Growth at room temperature (i.e., about 27° C.) was completed in about 8-10 days. Growth at about 55-60° C. resulted in the fungi never fully colonizing the media. High levels of humidity naturally occurred in all chambers and kept the materials from drying out during growth.

### Example 4

Bio-mat Production Using Neurospora crassa and Rhizopus oryzae species Cultures of Neurospora crassa, a filamentous fungus of the ascomycota, and Rhizopus oryzae, a filamentous fungus of the mucoromycete, were grown. At times, these two fungi were grown in liquid cultures. When the liquid cultures were allowed to sit unperturbed, it was observed that the fungi would form thick bio-mat films at the air-liquid interface of the still liquid media that grew thicker over time. The films of N. crassa were observed to have a leather-like or skin-like texture and elasticity, while the R. oryzae films were observed to resemble soaked cotton. Neurospora crassa is a strain of filamentous fungi that is known to regularly undergo anastomosis, which is a process that links mycelial networks together. Rhizopus oryzae also undergoes this anastomosis process, but because of the differences in the fungal biofilm properties, R. oryzae is thought to undergo anastomosis to a lesser degree compared to N. crassa. Experiments with various tested methods of culturing and processing of these films led to a system which produces textiles from fungal mycelial mats which have varying mechanical properties depending upon the species and conditions used. Some of the key advantages of the described production methods include the speed of production, the low cost of materials, the simplicity of the materials needed, and the variability in the final materials that can be created.

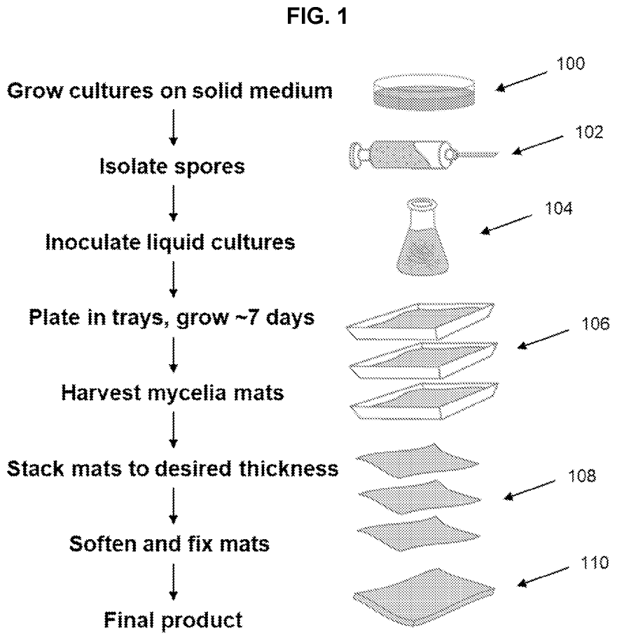

A general process flow for generating the textile material from filamentous fungi is illustrated in FIG. 1. Cultures of a specific filamentous fungal species are selected for the production of a textile material. The species selected can have a higher or lower rate of anastomosis, which will generate a more leather-like material or fibrous cotton-like material, respectively. The cultures are then grown on solid media in Petri dishes (100) in order to isolate conidia. The isolated conidia (102) are then used to create liquid cultures (104) which are grown in an approximately 1:10 ratio to the final culture volume used to create the mycelial mats. This ratio can be adjusted to increase or decrease the speed of mycelial mat production. The liquid cultures are grown in sterile Erlenmeyer flasks of the appropriate size (104), which are plugged with sterile cotton and placed on a rotor at 150 rpm. The resulting liquid cultures of mycelium are then homogenized using a sterilized blender. The homogenized cultures are then mixed with fresh liquid media and poured into sterilized metal trays with the desired length and width (106), accounting for the degree of shrinkage that occurs when the mycelial mats are processed. The final inoculated liquid sits 1 inch deep in the metal trays. The trays are then sealed with a lid or metal foil to protect from any contaminant growth and allowed to incubate for ˜7 days. At this point, culture conditions such as temperature and humidity can be adjusted to increase the growth rate of the material. Many trays are grown concurrently (106) so that a final textile of varying thickness can be created in a faster time.

Once the mycelial mats have formed a uniform film across the tray, the mycelial mats are gently harvested (108) and stacked together into one sheet (110). The final sheet of material is then softened using a plasticizer, such as vegetable glycerin, glycerol, or polyethylene glycol, before being chemically crosslinked using agents. The concentration of the plasticizer can be used to vary the stiffness of the final material, where higher concentrations lead to more malleable materials, and lower concentrations lead to stiff and hard materials. If a hard plastic-like material is desired, this can be generated using low plasticizer concentrations. Crosslinking agents that were found to work well with the generated fungal material include citric acid, calcium carbonate, paraformaldehyde, and vegetable tannins. Paraformaldehyde is a well-established chemical crosslinker of many biomolecules and has been used in leather manufacturing.

The final mycelial mat material can then be treated to create the desired surface properties, such as oiling for hydrophobicity, and dying for color variance. During experimentation, it was found that citric acid, calcium carbonate, and vegetable glycerin can be used to create a strongly crosslinked and malleable leather-like material, when using N. crassa as the starting culture. Additionally, 10% paraformaldehyde and polyethylene glycol were also observed to create a strongly crosslinked and malleable leather-like material with N. crassa as the starting culture. When using R. oryzae as the starting culture, it was found that the fibers of the mycelial mats could be easily pulled apart, suggesting that a variation of the process could be performed to create fibrous cotton-like materials such as threads or absorbent clothes.

### Example 5

### Growth Chambers and Inoculation Methods

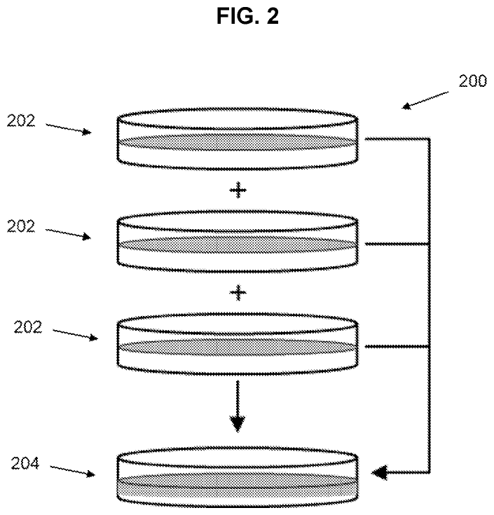

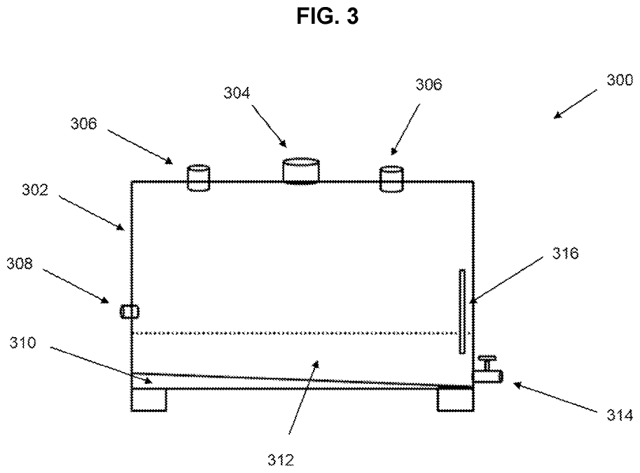

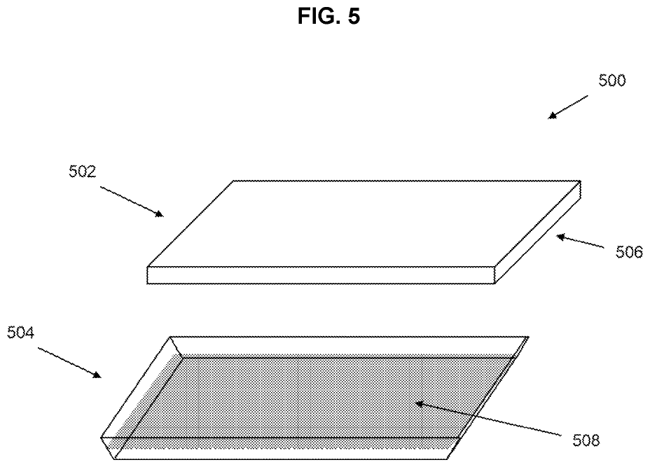

The original growth chambers used were sterile stacked petri dishes prepared in a sterile flow hood and sealed in paraffin after inoculation, as shown in FIG. 2 in a stacking petri dish method (200), as a “chamber precursor.” Specifically, a plurality of individual petri dish plates (202) was combined together to create a stacked fungal leather sample (204). This method could not be economically scaled up for larger sheets, however, so different growth chamber versions and inoculation methods were designed and tested. The chambers mainly needed to be cheap, sterilizable in an autoclave or an oven, and inert to the fungus. Several growth chamber versions were designed and tested, as shown in FIG. 3-5, but a stainless-steel baking sheet tray method (FIG. 5, “Chamber V3”) was found to generate the best results.

FIG. 3 shows a version 1 chamber model (“Chamber V1”) (300). The Chamber V1 model (300) includes a chamber body portion (302), a chamber cap portion including a cotton plug (304), a plurality of liquid media inlets (306), an aeration valve (308), a slanted bottom tray portion (310), a liquid media portion (312), a spray nozzle outlet (314), and a thermometer (316). This design was the first iteration that had spray nozzles (314) that allowed for media aerations, and a cap with a cotton plug that allowed for gas exchange (304). The bottom tray (310) was slanted to allow for easy draining and mycelial biomass extraction. This design was created before it was realized that a still liquid medium and a large surface area would yield large bio-mats without a need for aeration and drainage.

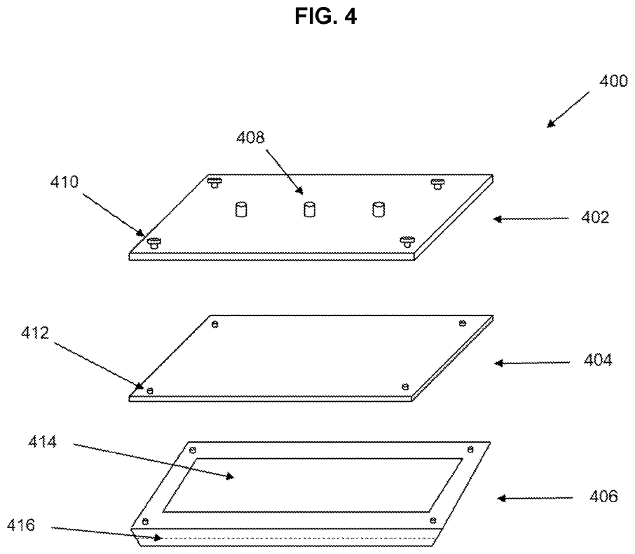

FIG. 4 shows a version 2 chamber model (“Chamber V2”) (400). The Chamber V2 model (400) includes a top layer portion (402), a middle layer portion (404), a bottom layer portion (406), a plurality of liquid media inlets (408), a plurality of layer connecting screws (410), a plurality of layer connecting apertures (412), and a liquid media portion (414) having a specific liquid media depth (416). This design was the first to actually be made and tested. It took longer to create large sheets on the still liquid with this design. The nozzles for liquid media addition that are shown in FIG. 4 (408) were not tested. Instead, the lid was modified to have cotton plug air inlets for gas exchange and the rim of the chamber was curved to overhang on the bottom tray to discourage contamination. This chamber version worked successfully, but usage indicated that a simpler chamber could be created.

FIG. 5 shows a version 3 chamber model (“Chamber V3”) (500). The Chamber V3 model (500) includes a top layer portion (502), a bottom layer portion (504), a top layer overhang portion (506), and a liquid media portion (508). This version includes a stainless-steel baking sheet tray method. It was simple to create, worked effectively, was cheap and scalable to any size needed, and generated consistent quality batches. The baking sheet tray (504) has a depth of about 2 inches throughout and is covered by a large flat stainless-steel lid (502) that has a 1-inch overhang over the sides of the tray (506) that discourages contamination. The chambers of this version did not have a tight seal and allowed enough necessary air gas exchange through the gaps in the tray, while the lid covered the media enough to protect from free-floating contaminants. The chamber trays were filled with media to a depth of about 1 inch. The length and width of the chamber trays could be of any size. The chamber media was then sterilized in an autoclave at 15 psi and 130° C. for 15 minutes or placed in a conventional oven at 85° C. for 30-45 minutes, which are standard sterilization conditions in fungal culture. The different inoculation methods used were direct inoculation with agar blocks, spore suspensions, and blended mycelial liquid cultures, as shown below in Table 6 with accompanying results.

TABLE 6
Inoculation Methods

| Method | Results |
|---|---|
| Agar blocks | A single agar block did not completely colonize the tray in any |
| | reasonable amount of time or before contamination occurred. However, |
| | an entire ~70 mm petri dish worth of agar blocks was able to |
| | successfully make a bio-mat. This indicated that agar blocks are not |
| | feasible but that the amount of starting inoculum was important. |
| Spore | The spore suspensions were able to successfully create bio-mats but |
| suspensions | took longer to grow and at times failed due to contamination. This |
| | indicated that established mycelial cultures were better suited for this |
| | method. |
| Blended liquid | This method consistently produced bio-mats of good quality, but the |
| mycelial cultures | amount of liquid culture needed still needed to be determined. To |
| | determine this, liquid cultures were grown for 48 hours from spore |
| | suspensions at room temperature, blended in a sterile blender, then |
| | added to the chambers at liquid culture inoculum: chamber media ratios |
| | of 1:100, 1:10, and 1:5. The 1:10 ratio was found to be sufficient to |
| | create bio-mats in a rapid amount of time while avoiding contamination. |
| | This entails 100 mL of inoculum for every liter of chamber media to |
| | create a bio-mat in 5-8 days at 40-45° C. The 1:5 dilution was faster |
| | and may be preferable in different circumstances. |

Aluminum trays were also found to work, but stainless steel was the most successful. When aluminum chamber trays were used, the fungi were able to metabolize the aluminum, which would damage the chambers over time. These chambers could easily be stacked in racks or on top of each other. The specific chamber size needs to be determined by the shrinkage of the bio-mat. In some instances, the bio-mat can shrink by approximately a third in area from the original tray area after processing into a leather-like fungal textile. Future chamber versions using this stainless-steel tray approach should include one or more drains.

### Example 6

Final Processing of Fungal Bio-Mats and Variation Between Rhizopus oryzae, Neurospora Crassa, and Agaricus bisporus

The bio-mats were grown in the version 3 chambers in yeast/malt extract broth liquid media at 40-45° C. for 5 days with a 1:10 starting ratio of liquid culture inoculum to media. The resulting bio-mats were then stacked and pressed together for a starting sheet that was 8 layers thick. The stack held together under its own adhesion and was processed further as one. The mat was then fixed with either 10% PFA or 10% citric acid with 5 g/L of CaCO₃ for 24 hours at room temperature. The resulting crosslinked bio-mats were pressed to remove the residual crosslinkers and then dried at 85° C. under a flat weight for another 12-24 hours. The bio-mat was then submerged in a 60° C. bath of 40% glycerol in DI water for 30 minutes to plasticize the bio-mat into a flexible leather-like material and was then air-dried at room temperature. The softening with plasticizer occurs on the combined stack of mats but because it is submerged in a 40% glycerol water solution, it is able to penetrate into the individual mat layers. When the stack of mats dry, the plasticizer is left in the material to maintain softness. The water in the plasticizer solution helps disrupt the hydrogen bonding and helps deliver the glycerol. The glycerol continues to disrupt bonding by getting between fibers of the mats and remains in the material after drying to maintain flexibility. The final leather was buffed with vegetable oil to create a hydrophobic surface. This process was done for bio-mats produced from both R. oryzae and N. crassa species.

For Neurospora crassa bio-mats, a material very similar to leather in appearance, texture and mechanical properties was created. The 8 layers of crosslinked bio-mat leather could not be ripped apart by hand and could be cut and pierced like leather. These material properties are thought to be due to the higher amount of anastomosis that Neurospora crassa undergoes.

For Rhizopus oryzae, a material similar to paper towel with a smooth surface texture on one side and a rough texture on the other was created. The material was very fibrous and easily tore apart into fibers. This version of the material may be kept hydrophilic, and minimally plasticized to encourage the paper-like aspects. Additionally, the fibers could be pulled apart and isolated into threads. This version of the R. oryzae material demonstrates that variation in fibrousness and interconnection between species is a tunable aspect of the bio-mats.

Agaricus bisporus chitin pulp was also explored. Chitin was first extracted from A. bisporus mushrooms. This precursor version of fungal textile created a different rubbery material that was like shoe soles or an eraser. This version of the material could likely be optimized to create a spongy, rubbery foam that may lend itself to molding. Variations in the use of the Alconox®, bleach, and NaOH extraction steps may also allow for foaming through high-speed blending in a manner similar to those used in algae-based biomaterials.

Furthermore, this version of the material could be used to create hard moldable materials for things like packaging if it is not processed further using crosslinkers and plasticizers. This would create a hard, porous plastic material. This harder version of the material was created during failed versions of the original attempt to create paper from the pulp. To create such a hard packaging material, one would first generate the final chitin extract pulp and press it into a mold similar to sheet metal forming, or injection molding, and allow it to dry at 85° C. before surface treatment. Shrinkage would need to be accounted for. Moreover, chitin has been known to have antibacterial properties which may lend it to food packaging applications.

### Example 7

Materials and Cost Analysis for Fungal-Based Paper Using Rhizopus oryzae and Agaricus bisporus

This analysis is based on a yield of 14 g of mycelial mass per liter of media containing 10 g fructose and 10 g of tomato paste. The method may be optimized using marmite and increasing fructose concentrations instead of using tomato paste.

Another way the process may be economically optimized is by pressing the papers into thinner layers and optimizing the number of sheets obtained from the mycelial mass.

TABLE 7
Reoccurring Cost for Mycelia Paper Production

| Component | Cost Per Unit | Bulk Cost | |
|---|---|---|---|
| Fructose | $0.0019/g | $50.99/5 | gal |
| Tomate paste | $0.0021/g | $37.84/18 | kg |
| Water | $0.0024 L | $0.01/1.1 | gal |
| Sodium hydroxide | $0.0024/g | $1.11/lb | |
| Acetic acid | $0.0083/mL | $157/5 | gal |
| Calcium carbonate | $0.00044/g | $9.95/50 | lb |
| Starch | $0.0044/g | $49.96/25 | lb |
| Glycerin | $0.0032/g | $73.50/50 | lb |

The cost of a sheet of 8.5×11-inch² uncoated paper with the current small-scale density and thinness is based on the below:

### Properties of Paper

Ratios: 75% processed chitin fibers, 10% CaCO₃, 10% corn starch, 5% vegetable glycerin
Density: 0.94 g/cm³
Thickness: 0.44 mm

### Extrapolating to Larger Paper Size

Area: 8.5×11 in =93.5 in²=603.2 cm²
Multiplied by the thickness: 0.044 cm=26.5 cm³

Multiply the extrapolated value by the measured density: 0.94 g/cm³×26.54 cm³=24.95 g of ready to use fungal paper pulp.

### Breaking Down to the Ratios in Grams:

Processed chitin fibers=18.7 g
CaCO₃=2.5 g
Corn starch=2.5 g
Glycerin=1.2 g

The cost of the chitin fibers includes the required 1 mL of 2.8 M sodium hydroxide per gram of raw fungal mycelia and 0.158 mL of acetic acid required for neutralization.

About 40% of the fungal mycelial mass is lost during the sodium hydroxide bath, so the required quantity of mycelia is: 0.6×(raw fibers) 18.7 g=31.2 g.

The amount of sodium hydroxide required is 3.5 g. The acetic acid needed to neutralize the sodium hydroxide is 4.9 mL. The cost of the media is $0.0414/L of media using 10 g fructose, 10 g tomato paste, and 1 L of water. The yield is 14 g/L of media. Collectively, the calculated costs are $0.04/L×1 L/14 g mycelia=$0.003/g of raw mycelia.

TABLE 8
Cost of Fungal-based 8.5 × 11-
inch Sheet of Uncoated paper (0.44 mm)

| | | |
|---|---|---|
| Material | Required Quantity | Cost per Unit |

| | | | |
|---|---|---|---|
| Mycelia | 31.2 | g | $0.1 |
| Sodium hydroxide | 3.5 | g | $0.009 |
| Acetic acid | 4.9 | mL | $0.05 |
| Corn starch | 2.5 | g | $0.01 |
| CaCO₃ | 2.5 | g | $0.001 |
| Glycerin | 1.2 | g | $0.004 |

| | |
|---|---|
| Total | $0.16 |

The cost may be decreased by reducing the thickness of the paper with the same or lower density. The media cost may be reduced, the yield of mycelial may be increased, and the cost of goods may be reduced by sourcing lower cost ingredients or purchasing in bulk quantities. For example, acetic acid and corn starch have the highest costs for the current formulation.

### Example 8

### Fungal Paper Process

An exemplary process for producing fungal paper material is outlined below:
(1) Grow fungal biomass;
(2) Macerate the fungal biomass into a pulp;
(3) Strain the pulp to remove liquid;
(4) Incubate the strained pulp in 1 mL of 2.8 M NaOH per gram of fungal biomass at room temperature for 30 min;
(5) Neutralize the hydroxide-treated pulp with 0.2 mL of acetic acid per mL of 2.8 M NaOH added;
(6) Strain the neutralized, NaOH-treated pulp to remove liquid;
(7) Wash the strained pulp with water in a 1:2 ratio (e.g., 100 mL of water per 200 g of pulp);
(8) Strain the washed pulp to remove wash water;
(9) Mix the strained pulp in a ratio of 75% pulp, 10% starch, 10% CaCO₃, 5% glycerol (e.g., vegetable glycerin);
(10) Press the mixture into sheets and dry at 85° C. for 2 hours;
(11) Optionally bleach; and
(12) Optionally cut to size.

What is claimed:

1. A method for generating a textile or paper material from filamentous fungi, the method comprising:
(a) growing a culture of filamentous fungi;
(b) forming a plurality of mycelial mats;
(c) harvesting the plurality of mycelial mats; and
(d) combining the plurality of mycelial mats into a stack of mycelial mats.

2. The method of claim 1, wherein growing the culture of filamentous fungi comprises first growing on solid media to isolate conidia, followed by growing the isolated conidia in liquid media to generate a liquid culture.

3. The method of claim 2, wherein forming the plurality of mycelial mats comprises inoculating liquid media in a plurality of growth chambers with the liquid culture.

4. The method of claim 3, wherein the liquid culture is added to the liquid media in the plurality of growth chambers at a 1:10 volume ratio.

5. The method of claim 3, wherein the liquid media comprises a broth comprising yeast extract and malt extract.

6. The method of claim 3, wherein the plurality of mycelial mats is formed on the liquid media in the growth chambers at a temperature ranging from about 25° C. to about 50° C. over about 5 days to about 10 days.

7. The method of claim 6, wherein the plurality of mycelial mats is formed on the liquid media in the growth chambers at a temperature ranging from about 40° C. to about 45° C. over about 5 days to about 8 days.

8. The method of claim 3, wherein each growth chamber comprises a stainless-steel chamber comprising a depth of about 2 inches.

9. The method of claim 8, wherein the growth chamber comprises about 1 inch of liquid media throughout the chamber.

10. The method of claim 1, wherein each of the mycelial mats comprises a dry thickness of up to about 1 mm.

11. The method of claim 1, wherein the filamentous fungi comprise Neurospora crassa or Rhizopus oryzae.

12. The method of claim 1, wherein the stack of mycelial mats comprises from about 2 to about 16 individual mycelial mats.

13. The method of claim 12, wherein the stack of mycelial mats comprises from about 8 to about 16 individual mycelial mats.

14. The method of claim 1, further comprising softening the stack of mycelial mats by treating with a plasticizer.

15. The method of claim 14, wherein treating with a plasticizer comprises submerging the stack of mycelial mats in a bath of plasticizer, wherein the plasticizer treats the outer surface of the stack and penetrates the interior of the stack to treat each of the plurality of mycelial mats.

16. The method of claim 15, wherein the bath of plasticizer comprises about 40% glycerol in water.

17. The method of claim 14, wherein a higher concentration of the plasticizer generates a softer and more malleable textile or paper material, and wherein a lower concentration of the plasticizer generates a harder and less malleable textile or paper material.

18. The method of claim 1, further comprising crosslinking the stack of mycelial mats using a crosslinking agent selected from the group consisting of citric acid, calcium carbonate, paraformaldehyde, vegetable tannins, a mixture of citric acid and calcium carbonate, and combinations thereof.

19. The method of claim 18, wherein the crosslinking agent comprises a mixture of citric acid and calcium carbonate comprising about 10% citric acid and about 5 g/L calcium carbonate.

20. The method of claim 1, further comprising processing the surface of the stack of mycelial mats to generate a textile or paper material with desired surface properties, wherein the desired surface properties include increased hydrophobicity or coloration.

21. The method of claim 20, wherein the surface of the stack of mycelial mats is treated with 100% vegetable oil to increase hydrophobicity.

22. The method of claim 20, wherein the surface of the stack of mycelial mats is treated with a color dye.

23. A textile or paper material from filamentous fungi made by the method of any one of claims 1-22.

24. A textile or paper material composition comprising a stack of filamentous fungi mycelial mats, made by a method comprising:
(a) growing a culture of filamentous fungi;
(b) forming a plurality of mycelial mats;
(c) harvesting the plurality of mycelial mats; and
(d) combining the plurality of mycelial mats into a stack of mycelial mats.

25. The composition of claim 24, wherein the filamentous fungi comprise Neurospora crassa or Rhizopus oryzae.

26. The composition of claim 24, wherein the textile material composition is leather-like when the filamentous fungi comprise Neurospora crassa.

27. The composition of claim 24, wherein the textile material composition is cotton-like when the filamentous fungi comprise Rhizopus oryzae.

28. The composition of claim 24, further comprising a plasticizer selected from the group consisting of mineral oil, vegetable oil, glycerol, and combinations thereof.

29. The composition of claim 24, further comprising a crosslinking agent selected from the group consisting of citric acid, calcium carbonate, paraformaldehyde, vegetable tannins, a mixture of citric acid and calcium carbonate, and combinations thereof.

30. The composition of claim 24, wherein the stack of filamentous fungi mycelial mats comprises from about 2 to about 16 individual mycelial mats.

31. The composition of claim 24, wherein the stack of filamentous fungi mycelial mats comprises a thickness ranging from about 2 mm to about 16 mm.

32. A fungal textile or paper material comprising a stack of mycelial mats formed from the species Neurospora crassa, wherein the stack of mycelial mats comprises a crosslinking agent and a plasticizer.

33. A fungal textile or paper material comprising a stack of mycelial mats formed from the species Rhizopus oryzae, wherein the stack of mycelial mats comprises a crosslinking agent and a plasticizer.

34. The fungal textile or paper material of claim 32 or 33, wherein the crosslinking agent comprises a mixture of citric acid and calcium carbonate.

35. The fungal textile or paper material of claim 32 or 33, wherein the plasticizer comprises glycerol.

36. The fungal textile or paper material of claim 32 or 33, wherein the stack of mycelial mats comprises a thickness ranging from about 2 mm to about 16 mm.

37. A growth chamber for cultivating fungi for producing mycelial mats, wherein the growth chamber comprises a stainless-steel chamber comprising a depth of about 2 inches, and wherein the growth chamber comprises about 1 inch of liquid media throughout the chamber.

38. A method for generating a fungal paper material from filamentous fungi, the method comprising:
(a) growing a fungal biomass of filamentous fungi;
(b) macerating the fungal biomass of filamentous fungi into a pulp;
(c) straining the pulp to remove liquid;
(d) incubating the pulp with NaOH;
(e) neutralizing the pulp with acetic acid;
(f) straining the pulp to remove liquid;
(g) washing the pulp with water;
(h) straining the pulp to remove liquid;
(i) mixing the pulp with starch, CaCO₃, and glycerol to create a mixture;
(j) pressing the mixture into one or more sheets of fungal paper material;
(k) drying the one or more sheets of fungal paper material;
(l) optionally, bleaching the one or more sheets of fungal paper material; and
(m) optionally, cutting the one or more sheets of fungal paper material.

39. The method of claim 38, wherein the fungal biomass of filamentous fungi comprises Neurospora crassa or Rhizopus oryzae.

40. The method of claim 38, wherein incubating the pulp with NaOH comprises incubating at room temperature for about 30 min.

41. The method of claim 38, wherein washing the pulp with water comprises a ratio of water:pulp of about 1:2.

42. The method of claim 38, wherein mixing the pulp with starch, CaCO₃, and glycerol comprises a mixture ratio of about 75% pulp, about 10% starch, about 10% CaCO₃, and about 5% glycerol.

43. The method of claim 38, wherein drying the one or more sheets of fungal paper material comprises drying at about 85° C. for about 2 hours.

44. A fungal paper material from filamentous fungi made by the method of any one of claims 38-43.
